(19) United States
(12) Patent Application Publication
(10) Pub. No.: US 2015/0073207 A1
(43) Pub. Date: Mar. 12, 2015

(54) SINGLE-PASS ENDOSCOPIC VESSEL HARVESTING

(71) Applicant: Terumo Cardiovascular Systems Corporation, Ann Arbor, MI (US)

(72) Inventor: Robert R. Langford, Eatonton, GA (US)

(21) Appl. No.: 14/021,537

(22) Filed: Sep. 9, 2013

(51) Int. Cl. A61B 17/32; A61L 27/36; A61F 2/06

(52) U.S. Cl. CPC A61B 17/320068; A61F 2/062; A61L 27/3625; A61B 17/320016; A61B 2017/320076; A61B 2017/00261
USPC 600/36

(57) ABSTRACT

An endoscopic harvesting device removes a vessel from a body. The vessel has an anterior side closest to the skin. A sheath extends in a longitudinal direction with a dissector tip for advancing along the vessel substantially along the anterior side to create a flanking tunnel spaced away from the vessel. A ring-shaped blade is mounted to the sheath and is disposed in a plane substantially perpendicular to the longitudinal direction and proximal of the dissector tip. The blade forms a lateral loop to encircle the vessel from the flanking tunnel and to make a vasiform cut including a pedicle around the vessel as the sheath advances.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to harvesting of living vessels for use in grafting, and, more specifically, to a harvesting device for endoscopically removing a vessel in a “no touch” condition with surrounding pedicle of fat and connective tissue.

Blood vessels are often dissected from one portion of a living body to be implanted in another portion of the body by a surgical procedure, such as in a coronary artery bypass graft (CABG) or other cardiovascular procedure. An artery or vein is “harvested” (i.e., removed) from its natural location in a patient's body and reconnected to provide blood circulation elsewhere in the body. Among the preferred sources for the vessels to be used as the bypass graft are the saphenous vein in the leg and the radial artery in the arm.

Endoscopic surgical procedures for harvesting a section of a blood vessel (e.g., the saphenous vein) subcutaneously have been developed in order to avoid disadvantages and potential complications of harvesting of the blood vessel by exposing the desired vein section externally through a continuous incision along the leg. The continuous incision for exposing the vein and for introducing the surgical instruments to seal and sever adjoining tissue and side branches of the vessel results in a significant healing process and associated risks.

The known minimally-invasive endoscopic techniques employ a small incision for locating the desired vessel and for introducing one or more endoscopic devices into the small incision. For example, typical commercially available products for performing the endoscopic blood vessel harvesting procedure include a number of separate endoscopic devices that are each inserted into the patient. These endoscopic devices include, for example, an insufflation mechanism having plastic tubing to supply air or CO₂ to insufflate the subcutaneous area; an endoscope having a camera and light cables in order to visualize both the dissection and harvesting procedures; a dissector mechanism to dissect or separate the vessel from connective tissues in the body; and a cutting mechanism to sever and seal any side branches from the vessel and then remove the vessel from the body. In certain instances, the combination of mechanisms can be bulky and cumbersome for the clinician performing the vessel harvesting. Also, in certain instances, these mechanisms require that a relatively large diameter wound and cavity be formed within the patient in order to accommodate all the separate mechanisms.

Existing harvesting devices have required an intricate and physically demanding procedure to isolate a vessel from surrounding tissue and to cut and coagulate side branches. This required a high level of skill and practice for the person performing the harvesting procedure. Even with good expertise, several potential sources of damage to the harvested vessel remain. Harvesting typically requires multiple passes of one or more separate devices resulting in much contact with the vessel, potentially leading to endothelial damage. To create a sufficient working space and to allow visualization for tissue separation and side branch cutting, significant insufflation is often used. The CO₂ insufflation gas can lead to tissue acidosis, CO₂ embolism, and other complications. The common use of electrocauterization for cutting and coagulating the side branches can result in thermal spreading to the harvested vessel and sometimes also results in side branch stubs that are too short for obtaining a good, leak-proof seal.

It has been discovered that improved patency can be obtained for a vein graph if some surrounding tissue is left intact around the desired vessel. However, conventional endoscopic devices have not been capable of maintaining a layer of surrounding tissue over the harvested vessel.

### SUMMARY OF THE INVENTION

The present invention provides a user-friendly device and procedure for endoscopically harvesting a vessel with a surrounding pedicle for use in grafting. Patency of the vessel is improved and trauma is reduced as a result of harvesting with no direct contact with the vessel. The simplified device and procedure achieve successful harvesting with less need for training or specialized skills.

In one aspect of the invention, an apparatus is provided for endoscopic harvesting of a vessel from a body. The vessel has an anterior side closest to the skin. A sheath extends in a longitudinal direction with a dissector tip for advancing along the vessel substantially along the anterior side to create a flanking tunnel spaced away from the vessel. A ring-shaped blade is mounted to the sheath and is disposed in a plane substantially perpendicular to the longitudinal direction and proximal of the dissector tip. The blade forms a lateral loop to encircle the vessel from the flanking tunnel and to make a vasiform cut including a pedicle around the vessel as the sheath advances.

### BRIEF DESCRIPTION OF THE DRAWINGS

FIG. 1 is an external view of a saphenous vein being harvested from a leg.

FIG. 2 shows a prior art dissector isolating a saphenous vein in an endoscopic procedure.

FIG. 3 is a partial cross section showing one embodiment of a harvesting device of the invention.

FIG. 4 is a side view of a blade member.

FIG. 5 is an end view of a blade member.

FIG. 6 is a perspective view of a blade member.

FIG. 7 is a perspective view of another blade member.

FIG. 8 depicts a blade ring about to be put in place around a vessel at an initial incision.

FIG. 9 depicts the blade ring after inserting it around a vessel at the initial incision and ready to make a vasiform cut.

FIG. 10 is a cross-sectional, subcutaneous view showing a harvested vessel with surrounding pedicle and flanking tunnel during harvesting.

FIG. 11 is a longitudinal cross section showing the harvesting of a vessel according to the present invention.

FIG. 12 illustrates a dissected pedicle ready for extraction from the body.

FIG. 13 is a cross section of a vessel illustrating the typical locations of side branches.

FIG. 14 illustrates a turning maneuver for severing and cauterizing a side branch being encountered near an anterior side of the harvested vessel.

### DETAILED DESCRIPTION OF PREFERRED EMBODIMENTS

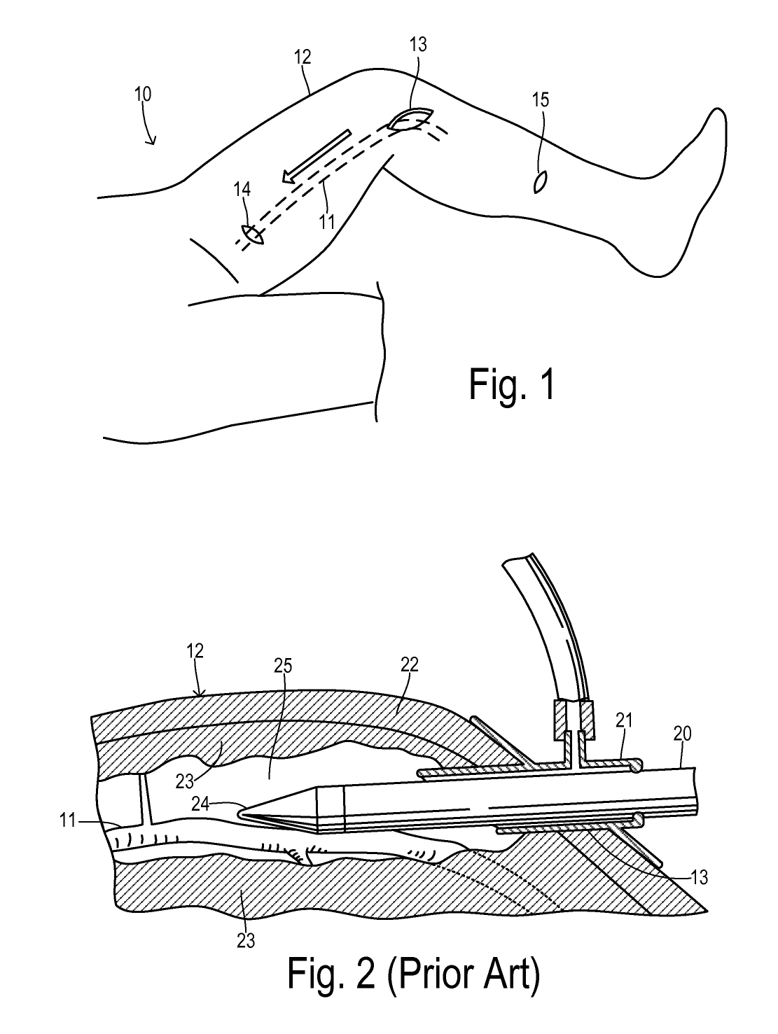

Referring now to FIG. 1, a patient 10 has a saphenous vein 11 within a lower limb 12. An incision 13 is made directly above vein 11, and tissue is peeled back from incision 13 to access the vein. Endoscopic instruments have been inserted through incision 13 to separate vein 11 from connective tissue and then to sever and cauterize side branches that extend from vein 11. A second incision or stab wound 14 is created at a second position on limb 12 so that a second end of vein 11 can be severed. Vein 11 is then extracted through one of the incisions. The entry point or second incision can be placed at various locations along vein 11 as shown at 15.

FIG. 2 shows a prior art dissector rod 20 inserted into a body through a trocar 21 which maintains an opening at incision 13 and provides a seal for insufflation gas. Trocar 21 may be adhesively attached to skin 22. Subcutaneous tissue 23 is peeled opened using a transparent tip 24 on dissector rod 20 to create an interior cavity 25 around saphenous vein 11. Insufflation gas may be introduced into cavity 25 (e.g., via appropriate tubing to trocar 21 or via a channel within rod 20) in order to maintain visualization of vein 11 via an endoscopic camera provided within dissector 20 as is known in the art. Dissector rod 20 may be removed after exposing vein 11 so that a cutting instrument can be reinserted for cutting and coagulating the side branches. By the time the section of vein 11 is removed, it has been subjected to handling that potentially causes endothelial and/or thermal injury.

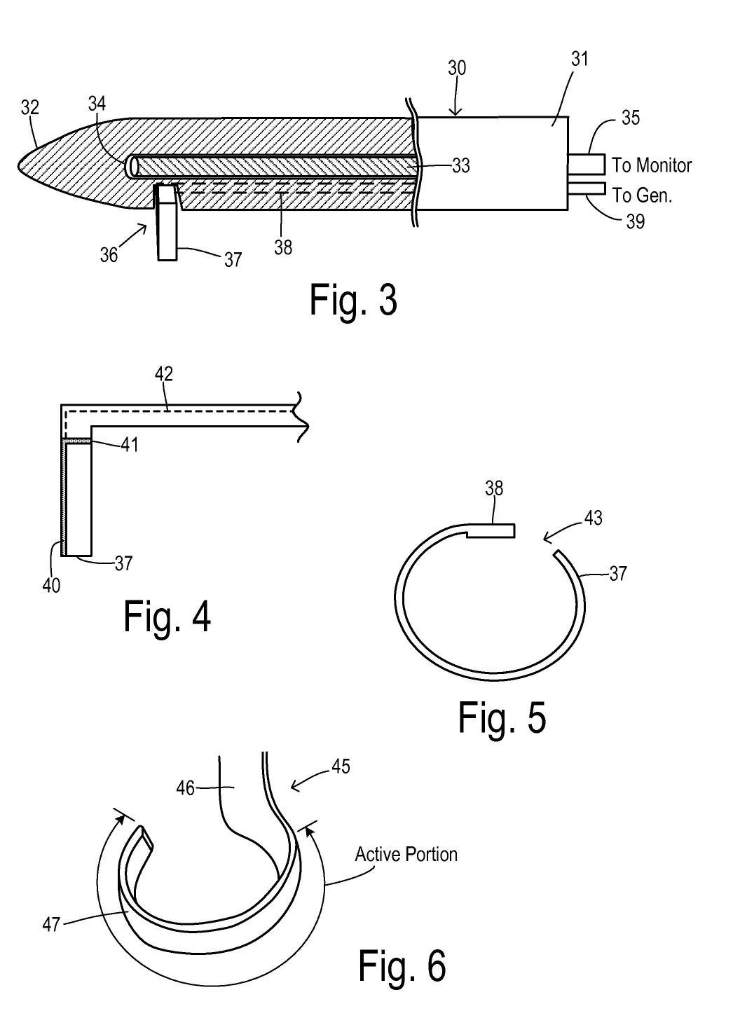

An improved endoscopic surgical instrument for harvesting a vessel in a single pass and with an intact pedicle around the vessel is shown in FIG. 3. A device body or sheath 30 has a handle 31 at one end and a dissector tip 32 at the other end. An endoscope element 33 is contained within a receptacle 34 within sheath 30 having a forward view through tip 32 (i.e., at least a portion of tip 32 is preferably made of a transparent material). Endoscope element 33 has an end connection 35 connected to a monitor and light source as is known in the art.

A cutter element 36 assembles into a corresponding groove(s) on sheath 30. Element 36 has an intermediate section 38 connected at one end to a cutting blade 37 which is positioned proximally of tip 32. Blade 37 is disposed in a plane substantially perpendicular to the longitudinal direction of sheath 30, and is the only ultrasonically-active portion of cutter element 36. At the other end of intermediate section 38, a connector 39 is provided for coupling to a power source which may preferably be an ultrasonic generator, for example. Other energy sources could also be used. Intermediate section 38 nests together with sheath body 30 in order to provide a generally smooth outer surface of the combined instrument. Preferably, no insufflation port is constructed in the harvester since no CO₂ insufflation is necessary.

Blade 37 is ring-shaped and may perform any type of cutting, such as electro-cautery or ultrasonic. In a preferred embodiment, ultrasonic cutting and cauterization may be used. As shown in FIG. 4, ring-shaped blade 37 may have a leading edge 40 and a terminal edge 41 made of a piezoelectric material of the type conventionally used in ultrasonic surgical instruments. Signal transmission lines 42 are preferably disposed on intermediate element 38 to couple a desired oscillating signal to the piezoelectric material when cutting is to be performed. By avoiding the generation of any significant heat, thermal spreading to the target vessel is avoided. As shown in the end view of FIG. 5, blade 37 preferably forms an incomplete ring resulting in a gap 43 between terminal end 41 and intermediate section 38. The ring is not complete around the circumference so that the vessel can be loaded within the ring as a first step, resulting in the vessel and surrounding pedicle being centered in the ring. Then the ring and its support/power supply section can be attached to the harvester rod, forming one integral piece that can be manually guided to dissect the vessel/pedicle.

FIG. 6 illustrates another embodiment of a ring-shaped blade 45 having an intermediate section 46 transitioning to a curved end 47. Only curved end 47 is ultrasonically active. Preferably, a variety of sizes and shapes are made available to the user in order to adapt an instrument to the size and/or location of a target vessel being harvested from a particular patient.

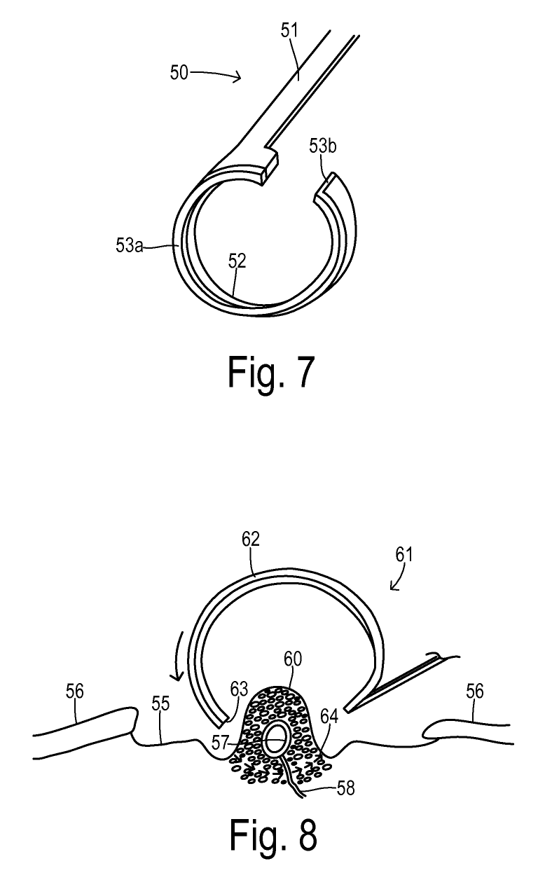

FIG. 7 shows yet another embodiment of a cutting element 50 having an intermediate section 51 supporting a ring blade 52. A narrow leading edge of ring blade 52 is comprised of an ultrasonic material 53A, while an open end 53B likewise contains the ultrasonic material for performing tissue cutting during initial placement of ring blade 52 around a vessel as described below.

As shown in FIG. 8, an incision has been made through a skin layer 56 to internal tissue 55 in order to obtain access to a tissue region 60 around a target vessel 57. An anterior side of vessel 57 (i.e., toward the exterior or skin side) is mostly free of side branches. Thus, a side branch 58 extends deeper into tissue 55 away from the anterior side. A cutting element 61 has a ring blade 62 with an open end 63 which is used to cut beneath target vessel 57 so that ring blade 62 may be placed as a lateral loop encircling vessel 57. Tissue 60 around vessel 57 is preferably pinched upward with a grasping tool (not shown) in order to facilitate entry of open end 63 along an incision path 64. Initial placement of ring blade 62 into path 64 may be done prior to or after the installation of cutting element 61 onto the sheath body.

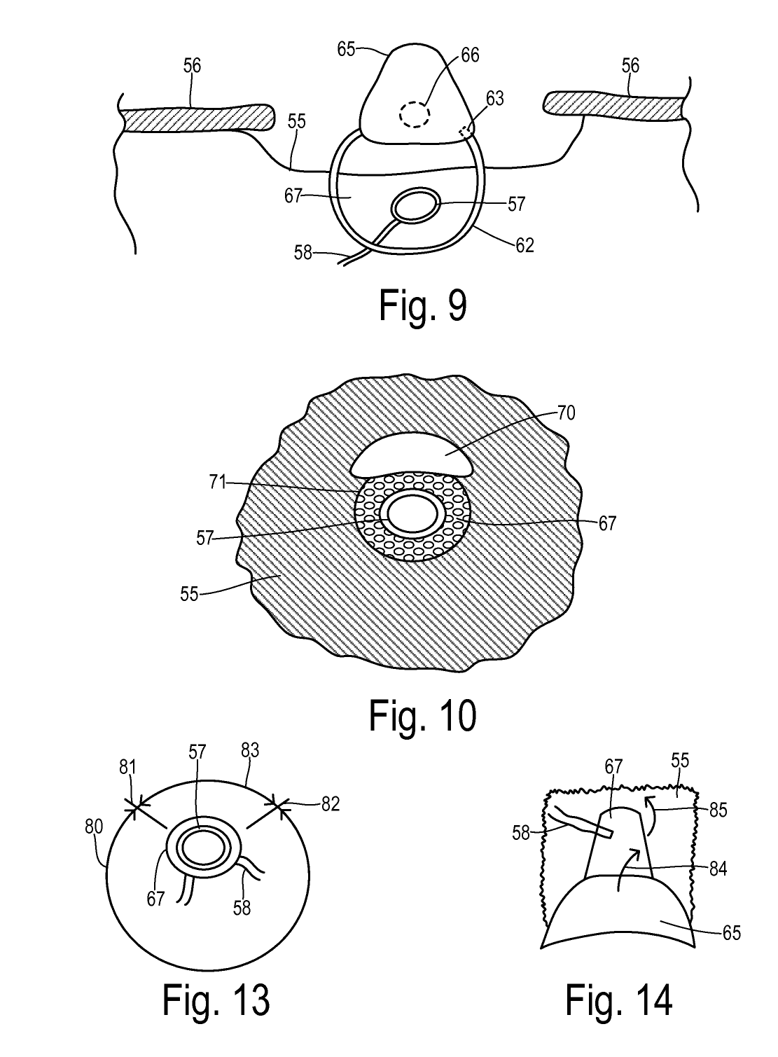

FIG. 9 shows ring blade 62 in its initial position around target vessel 57 with ring blade 62 installed onto dissector tip 65. An endoscope lens 66 incorporated in tip 65 may be used to visualize the tissue around and including target vessel 57. A tissue section 67 within the perimeter of ring blade 62 makes up a pedicle around vessel 57. With the device in the initial position shown in FIG. 9, vessel 57 remains fully intact (i.e., its end has not yet been cut). By maintaining the integrity of vessel 57 and its surrounding pedicle 67 when blade 62 is advanced, a counter-traction is maintained when tip 65 performs its cutting action along vessel 57 as described below.

Once cutting element 61 is assembled onto the sheath body, open end 63 of blade 62 is preferably positioned in an open space inside the general profile of the sheath body. This ensures that all the tissue to be cut is contacted by the active region of the ring. Open end 63 does not actually contact tip 65, but is instead free to vibrate ultrasonically in response to the energy source.

With ring blade 62 encircling target vessel 57 at the initial incision, dissector tip 65 is advanced along the vessel substantially along the anterior side in order to create a flanking tunnel 70 spaced away from vessel 57 as shown in FIG. 10. As an operator presses the handle forward and guides dissector tip 65 just slightly above the position of target vessel 57, a vasiform cut 71 is made encircling a pedicle 67 and vessel 57. The length of vasiform cut 71 progresses as dissector tip advances for a desired length of vessel to be harvested. FIG. 11 shows a side view during the formation of a vasiform cut around target vessel 57. As the operator advances tip 65 above target vessel 57 so as to maintain an amount of connective tissue 55 between tip 65 and vessel 57, ring blade 67 is energized in order to make the vasiform cut that simultaneously excises pedicle 67 and vessel 57 while automatically severing and cauterizing side branches such as branches 73 and 74. Thus, only a single pass is needed in order to form the desired pedicle with embedded vessel, thereby resulting in minimal disturbance or injury to target vessel 57.

As shown in FIG. 12, once a sufficient length of pedicle 67 containing the target vessel has been made between an initial end 76 and a final end 77, a second incision 78 is made through skin 56 in order to access final end 77. A scalpel or other cutting instrument can then be used at each incision in order to sever the ends of pedicle 67 so that the harvested vessel can be removed and the remaining ends of the saphenous vein can be sealed off. Thereafter, instrument 30 may be removed from the patient.

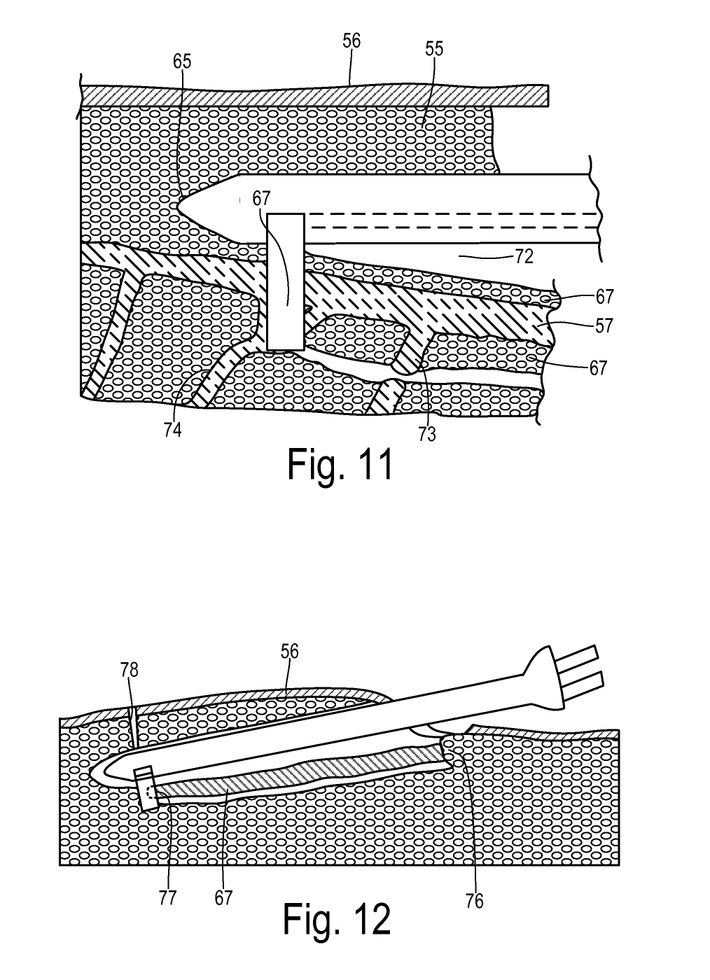

FIG. 13 shows a transverse cross-section through vessel 57 and pedicle 67. An anterior side 83 of vessel 57 is located between radial positions 81 and 82. This anterior side 83 is generally free from side branches. Nearly all the side branches, such as side branch 58, are encountered at a non-anterior side 80 between radial positions 81 and 82. By configuring the ring blade to span non-anterior portion 80 and to have each end of the ring contained within the flanking tunnel, a procedure is obtained wherein most all side branches are automatically cut and cauterized without the operator taking any special actions. In the event that a side branch is encountered along the anterior side as shown in FIG. 14, the operator may rotate tip 65 in a direction away from a side branch 58 as shown by arrow 84 in order to avoid contact between dissector tip 65 and branch 58. This places branch 58 in a position to be cut by the ring blade. As tip 65 advances past the position of side branch 58, the operator restores the handle to the original orientation, thus bringing tip 65 back to the original anterior side as shown by arrow 85.

Using the foregoing invention, a target vessel can be gently retracted and may be harvested together with a surrounding pedicle that provides protection and life support for the vessel. No CO₂ is needed, although a small amount of CO₂ insufflation may be used if desired, but may be performed at a significantly reduced amount as compared to the prior art. A speed of forward movement of the dissector tip and ring blade may be adjusted by the operator according to any variations in the tissue as they advance. In addition, a magnitude of ultrasonic oscillation can be adjusted by the operator to ensure adequate coagulation and ligation of a range of small to larger side branches. A passive or low suction incorporated in the harvester could also be used in order to remove any desiccation fluid created by the ring activity plus any smoke that may need to be vented. At no point in the inventive procedure is the vessel “skeletonized”. The anterior pass of the dissector tip is steered so that several cell layers of fat and connective tissue remain attached to the vessel. Visualization of the vessel through the thin layers is sufficient to steer the instrument and anticipate the vessel's path.

What is claimed is:

1. Apparatus for endoscopic harvesting of a vessel from a body, wherein the vessel has an anterior side closest to the skin, comprising:
a sheath extending in a longitudinal direction with a dissector tip for advancing along the vessel substantially along the anterior side to create a flanking tunnel spaced away from the vessel; and
a ring-shaped blade mounted to the sheath and disposed in a plane substantially perpendicular to the longitudinal direction and proximal of the dissector tip, wherein the blade forms a lateral loop to encircle the vessel from the flanking tunnel and to make a vasiform cut including a pedicle around the vessel as the sheath advances.

2. The apparatus of claim 1 wherein the vessel has a plurality of side branches, and wherein the blade has a leading edge that cuts and cauterizes the side branches as the sheath advances.

3. The apparatus of claim 2 wherein the blade comprises an ultrasonic cutter at the leading edge.

4. The apparatus of claim 1 wherein the blade is comprised of a partial ring with an open end of the lateral loop being adapted to be initially inserted around the vessel by rotating the blade to puncture tissue around the vessel and the surrounding pedicle, so that the vessel remains unsevered while the vasiform cut is made.

5. The apparatus of claim 1 further comprising an endoscopic lens secured to the sheath for providing a view of the vessel.

6. A method of harvesting a blood vessel from a body comprising the steps of:
making an initial incision to visualize a target vessel;
perforating tissue surrounding the vessel to loop a ring blade around the vessel;
advancing a dissector tip at an end of a longitudinal sheath to dissect a flanking tunnel substantially along an anterior side of the vessel, wherein the ring blade is fixed to the sheath in a plane substantially perpendicular to the sheath and proximal of the dissector tip so that the ring blade simultaneously makes a vasiform cut including a pedicle surrounding the vessel;
after advancing the dissector tip to a finishing location, severing the vessel at the initial incision and at the finishing location; and
extracting the vessel and pedicle from the body.

7. The method of claim 6 wherein if a side branch of the target vessel is encountered on the anterior side while advancing the dissector tip, then further comprising the steps of:
steering the flanking tunnel circumferentially away from the side branch while continuing to advance the dissector tip; and
steering the flanking tunnel back to the anterior side after the side branch is cut by the ring blade.

8. The method of claim 6 wherein the ring blade is an ultrasonic cutter.

9. The method of claim 6 further comprising the step of:
attaching the ring blade to the sheath after looping around the vessel.
